(19) United States
(12) Patent Application Publication
(10) Pub. No.: US 2011/0178468 A1
(43) Pub. Date: Jul. 21, 2011

(54) MEDICINAL FLUID INJECTOR WITH BACKFLOW PREVENTION FUNCTION

(75) Inventor: Jong-Woo LEE, Seoul (KR)

(73) Assignee: ACE MEDICAL CO., LTD., Gyeonggi-Do (KR)

(21) Appl. No.: 12/942,241

(22) Filed: Nov. 9, 2010

(30) Foreign Application Priority Data
Jan. 19, 2010 (KR) 20-2010-0000586

(51) Int. Cl. A61M 5/00

(52) U.S. Cl. 604/186

(57) ABSTRACT

A medicinal fluid injector having branch lines to inject a ringer solution together with a treatment solution is provided. If the vein of the patient is clogged, the treatment solution is not injected into the patient, but flows back into the ringer solution, which is injected into the patient through a free fall scheme. If the vein is open, the medicinal fluid is excessively injected at a time, causing fatal accident to the patient. To solve this problem, a check valve is installed in an injection tube to prevent the medicinal fluid or the blood from flowing back into the ringer solution even if pressure difference occurs in the medicinal fluid injector. If the medicinal fluid stagnates, an alarm is generated to notify medical staffs of the emergency, so that medicinal fluid injector is safely used.

### PRIORITY

This application claims the benefit under 35 U.S.C. § 119(a) of a Korean patent application filed on Jan. 19, 2010 in the Korean Intellectual Property Office and assigned Serial No. 20-2010-0000586, the entire disclosure of which is hereby incorporated by reference

### BACKGROUND OF THE INVENTION

1. Field of the Invention

The present invention relates to a medicinal fluid injector having branch lines to inject a ringer solution together with a treatment solution. More particularly, the present invention relates to a medicinal fluid injector, which can be safely used by preventing a treatment solution forcibly injected by a mechanical injection device from flowing back into a ringer solution and generating an alarm sound when a vein of a patient clogs.

2. Description of the Related Art

In general, a ringer solution is injected into a patient treated in a hospital for the purpose of post-treatment and nutrition feeding, and a treatment solution including specific medicines is injected into the patient to protect and treat the patient or to attenuate the pain. If the ringer solution is injected into the patient separately from the treatment solution, two catheters are required to inject the medicinal fluid into the patient. Since a plurality of catheters is used to inject the medicinal fluid, a skin of the patient may be damaged and medical staffs may feel inconvenience when injecting the medicinal fluid into the patient.

Recently, there has been suggested a method of simultaneously injecting the ringer solution and the treatment solution by using a medicinal fluid injector having branch lines. However, since a fixed quantity of the treatment solution must be periodically injected into the patient on time, the treatment solution may be forcibly injected into the patient by a mechanical injection device or an electrical injection device. At this time, if the vein of the patient clogs, the treatment solution is not injected into the patient, but flows back into the ringer solution injected into the patient through a free fall scheme. Thus, actually, the treatment solution, which is necessarily required for the patient, may not be injected into the patient.

In addition, if the medicinal fluid stagnates without being injected into the patient, the injection device must raise an alarm. However, the injection device may not raise an alarm even if the medicinal fluid flows back into the ringer solution without being injected into the patient.

Therefore, if the medical staffs do not urgently deal with this situation, the serious medical accident may occur.

In particular, an unconscious patient may not feel the pain or may not express the pain even if he/she sometimes feels the pain. In addition, the unconscious patient cannot check the treatment status and cannot notify the present status to another person even if the abnormal symptom is found in the body of the patient or the medicinal fluid or the blood flows back due to the clogging of the vein. For this reason, the medical staffs must frequently check the status of the patient, so many medical staffs must be supplemented to check the status of the patient. However, this system is very inefficient and may not be a final solution for ensuring the safety of the patient.

### SUMMARY OF THE INVENTION

An aspect of the present invention is to address at least the above-mentioned problems and/or disadvantages and to provide at least the advantages described below. Accordingly, an aspect of the present invention is to provide a medicinal fluid injector, in which a ringer solution and a treatment solution are input into input tubes having branch lines such that the ringer solution and the treatment solution can be injected into a patient through an integral injection line while preventing the treatment solution from flowing back into the ringer solution, and an alarm is generated when the injection of the medicinal fluid is forcibly stopped due to the clogging of a vein of the patient, thereby enabling medical staffs to urgently deal with the emergency.

In accordance with an aspect of the present invention. A medicinal fluid injector with a backflow preventing function is provided. The medicinal fluid injector includes a branch member having two input tubes and one discharge tube, and a medicinal fluid injection tube connected to a lower end of the branch member through a hose and coupled with a catheter. A backflow prevention device is provided in the input tube receiving the ringer solution in such a manner that a medicinal fluid may flow in one direction according to the bending of a packing, thereby preventing the treatment solution, which is not injected into the patient, from flowing back into the ringer solution through the ringer input tube.

As described above, the ringer solution and the treatment solution can be injected into the patient through the branch member having the integral injection line, so that the medicinal fluid injector can be conveniently used. In addition, since plural catheters are not necessary, the skin of the patient can be prevented from being damaged. Further, the medicinal fluid or the blood can be prevented from flowing back into the ringer solution and the alarm is generated from the injection device when the medicinal fluid stagnates, so that the medicinal fluid injector can be safely used when injecting the medicinal fluid into the unconscious patient.

Other aspects, advantages, and salient features of the invention will become apparent to those skilled in the art from the following detailed description, which, taken in conjunction with the annexed drawings, discloses exemplary embodiments of the invention.

### BRIEF DESCRIPTION OF THE DRAWINGS

The above and other aspects, features, and advantages of certain exemplary embodiments of the present invention will be more apparent from the following description taken in conjunction with the accompanying drawings, in which:

FIG. 1 is a perspective view showing a medicinal fluid injector according to an exemplary embodiment of the present invention;

FIG. 1A is an exploded perspective view showing a structure of a check valve according to an exemplary embodiment of the present invention;

FIG. 2 is a front view showing a medicinal fluid injector according to an exemplary embodiment of the present invention;

FIGS. 3A and 3B are sectional views showing a flow of medicinal fluid in a check valve according to an exemplary embodiment of the present invention;

FIGS. 4A and 4B are sectional views showing a structure of a check valve according to an exemplary embodiment of the present invention;

FIG. 5 is a perspective view showing a usage state of a medicinal fluid injector according to an exemplary embodiment of the present invention; and

FIG. 6 is a schematic view showing a medicinal fluid tube according to the related art.

Throughout the drawings, it should be noted that like reference numbers are used to depict the same or similar elements, features, and structures.

### DETAILED DESCRIPTION OF THE EXEMPLARY EMBODIMENTS

The following description with reference to the accompanying drawings is provided to assist in a comprehensive understanding of exemplary embodiments of the invention as defined by the claims and their equivalents. It includes various specific details to assist in that understanding but these are to be regarded as merely exemplary. Accordingly, those of ordinary skill in the art will recognize that various changes and modifications of the embodiments described herein can be made without departing from the scope and spirit of the invention. In addition, descriptions of well-known functions and constructions may be omitted for clarity and conciseness.

The terms and words used in the following description and claims are not limited to the bibliographical meanings, but, are merely used by the inventor to enable a clear and consistent understanding of the invention. Accordingly, it should be apparent to those skilled in the art that the following description of exemplary embodiments of the present invention is provided for illustration purpose only and not for the purpose of limiting the invention as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

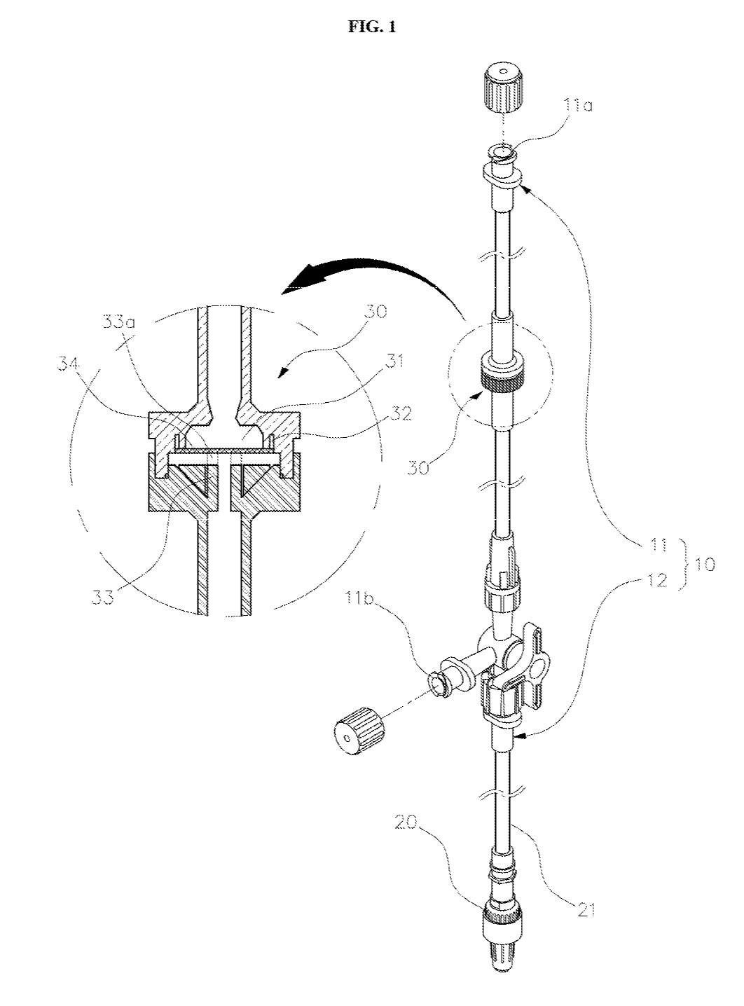

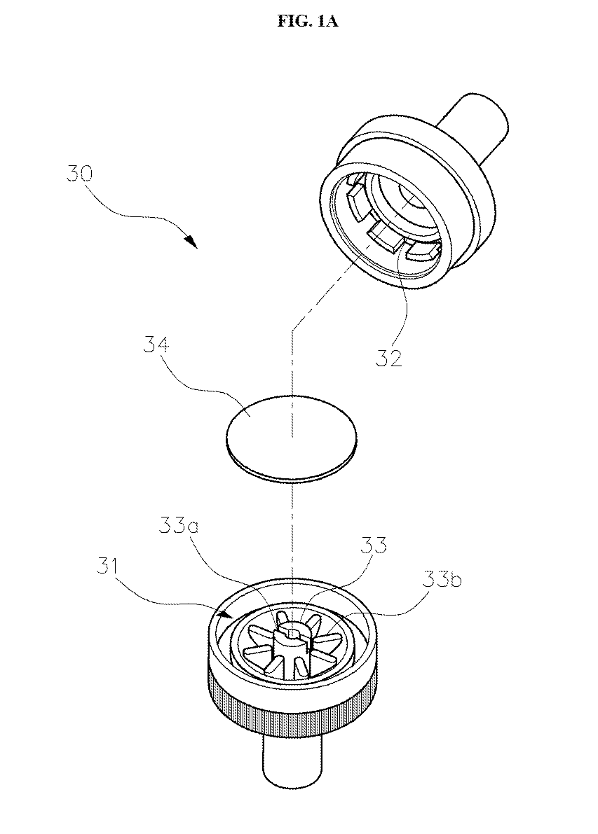

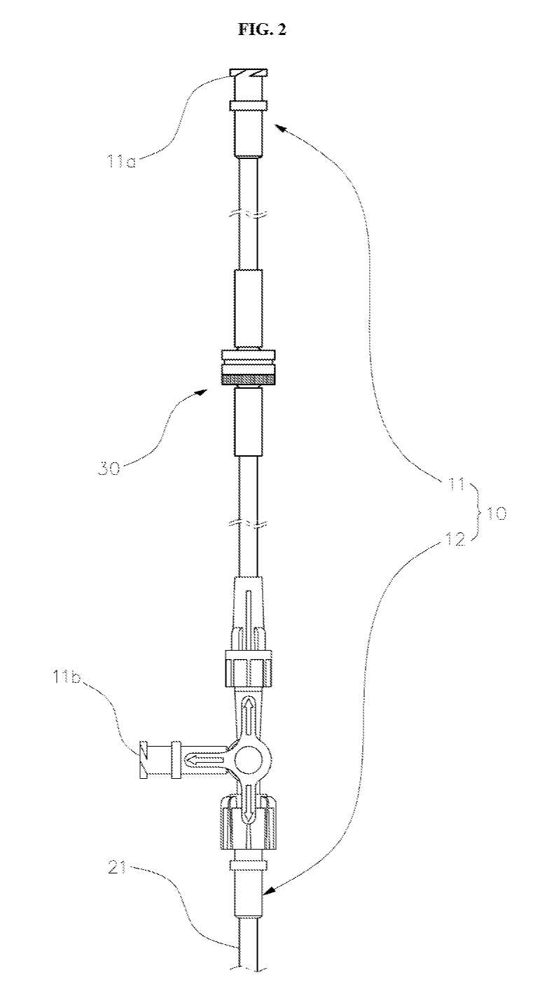

FIG. 1 is a medicinal fluid injector according to an exemplary embodiment the present invention.

Referring to FIG. 1, the medicinal fluid injector includes a branch member 10 including a plurality of input tubes 11 and one discharge tube 12 and an injection tube 20 connected to a lower end of the discharge tube 12 through a hose 21 and coupled with a catheter 100. A check valve 30 is provided in the input tube 11 in such a manner that a medicinal fluid is discharged through the check valve 30 while preventing the medicinal fluid or the blood from flowing back.

The input tubes 11 are divided into a ringer solution input tube 11a and a treatment solution input tube 11b, which are converged at the discharge tube 12, in order to inject the ringer solution and the treatment solution into the patient for the nutrition feeding and the treatment for the patient.

The ringer solution input tube 11a is connected to a ringer bottle such that the ringer solution can be supplied into the ringer solution input tube 11a through the free fall scheme, and the treatment medicine or the anodyne is supplied into the treatment solution input tube 11b. A fixed quantity of the treatment medicine or the anodyne must be continuously supplied, so the treatment medicine or the anodyne may be forcibly fed by a mechanical device or an electrical device.

In detail, as the medicinal fluid is supplied into an elastic bulb by an infusion pump, a small-sized motor or a rubber member, the elastic bulb is compressed and expanded such that a fixed quantity of the medicinal fluid can be supplied through a capillary tube. At this time, since the medicinal fluid is forcibly supplied into the treatment solution tube 11b through the compression of the elastic bulb, the medicinal fluid may not flow back, so an additional backflow prevention device is not necessary in the treatment solution input tube 11b. Thus, the check valve 30 is installed only in the ringer solution input tube 11a.

The check value 30 has a tubular body formed at the center thereof with an expansion space 31 for accommodating a packing 34 therein. An edge protrusion 32 protrudes downward from a top surface of the expansion space 31 and a protrusion tube 33 protrudes upward from the center of a bottom surface of the expansion space 31. The center of the packing 34 installed in the expansion space 31 is supported by the protrusion tube 33 and an edge of the packing 34 fixedly adheres to the edge protrusion 32.

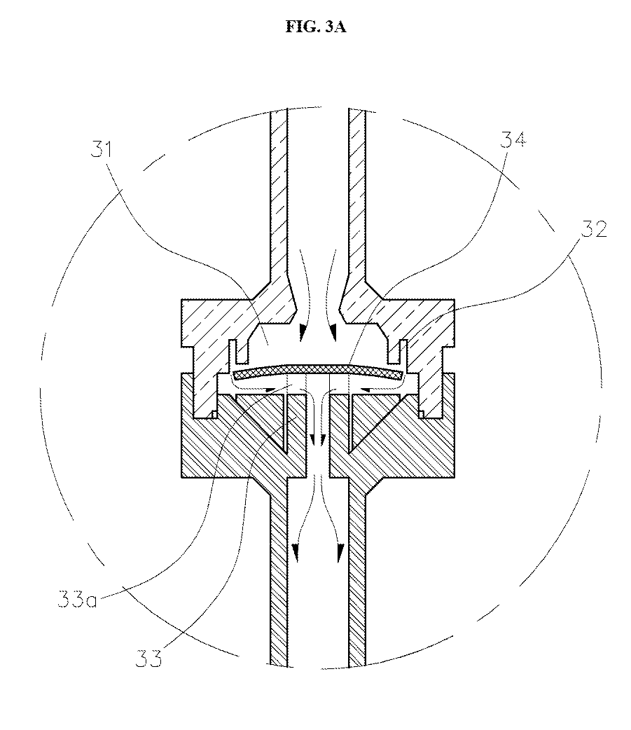

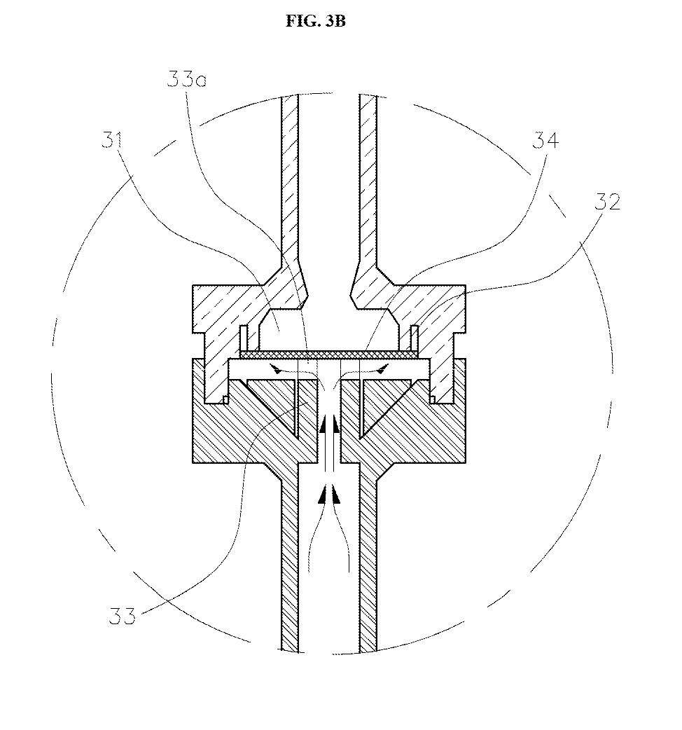

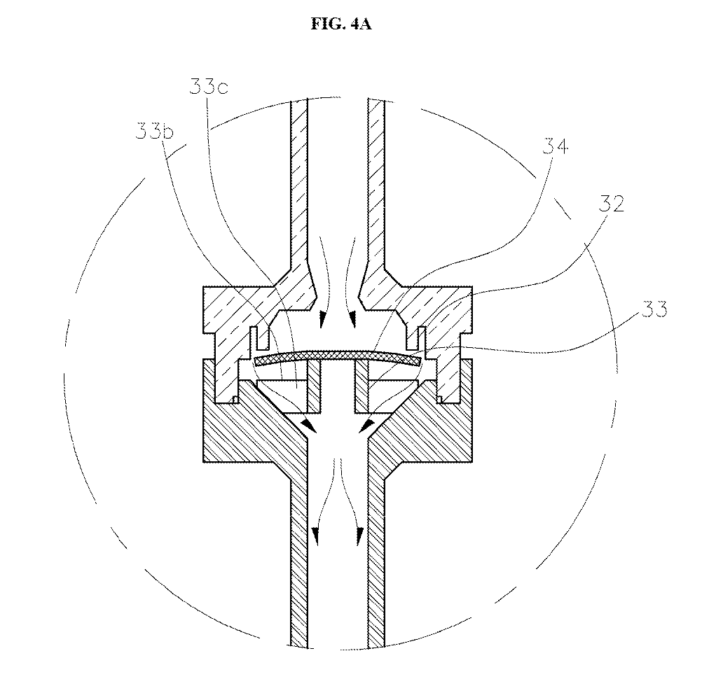

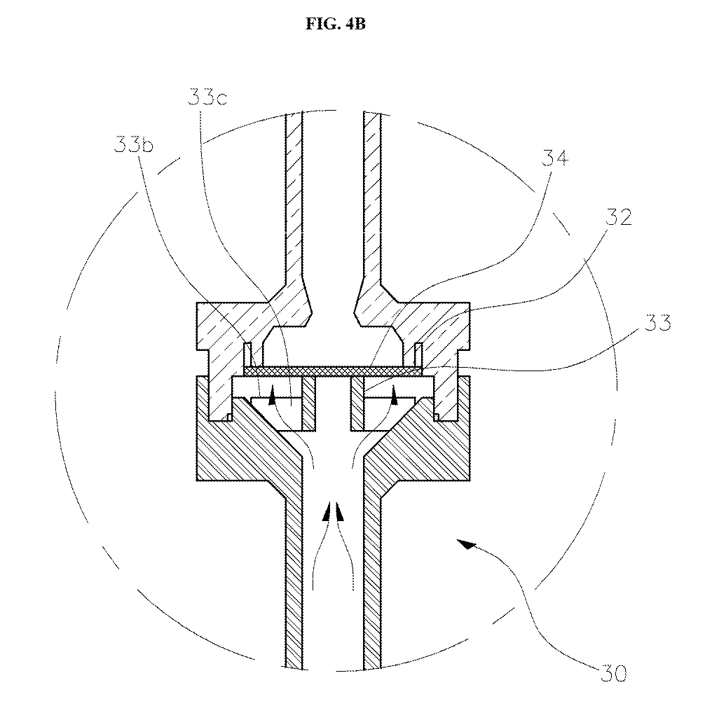

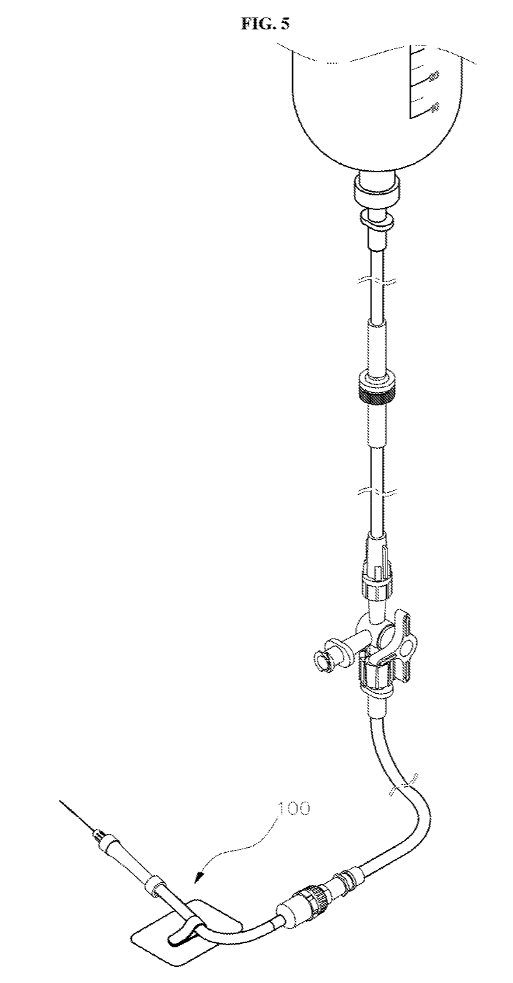

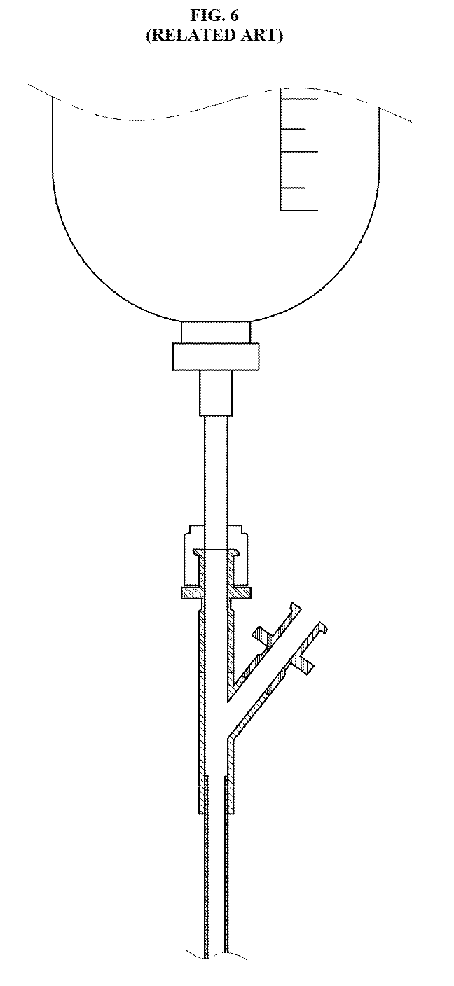

Therefore, the packing 34 is movable according to the pressure of the medicinal solution. As shown in FIGS. 3A and 3B, when the pressure is applied downward from the top of the check valve 30, the edge of the packing 34 is bent downward to open a fluid path. In addition, when the pressure is applied upward from a bottom of the check valve 30, the edge of the packing 34 adheres to the edge protrusion 32 to close the fluid path.

In detail, if the medicinal fluid supplied through the ringer solution input tube 11a is filled in the expansion space 31, the internal pressure is generated in the expansion space 31. At this time, the edge of the packing is elastically bent downward in a state in which the center of the packing 34 is supported by the protrusion tube 33, so that the expansion space 31 is open. Thus, the medicinal fluid can be discharged downward through a gap formed between the expansion space 31 and the edge protrusion 32.

In contrast, if the pressure below the check valve 30 is higher than the pressure above the check valve 30, that is, if the high pressure is generated in the vicinity of the discharge tube, the medicinal fluid or the blood may flow up toward the ringer solution. In addition, if there is variation of the pressure in the ringer solution being fed under the constant pressure, the packing 34 adheres to the edge protrusion 32 of the expansion space 31 to close the expansion space 31, thereby preventing the treatment solution or the blood from flowing back toward the ringer solution. Thus, a fixed quantity of the treatment medicine or the anodyne can be smoothly injected into the patient without being leaked.

As shown in FIGS. 1A, 3A and 3B, the protrusion tube 33 is formed at an outer peripheral surface of an upper end thereof with an incised slot 33a to form the fluid path of the check valve 30. In addition, an opening hole 33c can be formed below blades 33b, which are radially arranged along the outer peripheral surface of the protrusion tube 33, to form the fluid path of the check valve 30 to discharge the medicinal fluid.

While the invention has been shown and described with reference to certain exemplary embodiments thereof, it will be understood by those skilled in the art that various changes in form and details may be made therein without departing from the scope and spirit of the invention as defined by the appended claims and their equivalents.

What is claimed is:

1. A medicinal fluid injector with a backflow preventing function, the medicinal fluid injector comprising:
a branch member including an input tube divided into a ringer solution input tube and a treatment solution input tube, and a discharge tube coupled with the input tube; and
an injection tube connected to the discharge tube through a hose and coupled with a catheter, wherein
a check valve is provided in the ringer solution input tube in such a manner that a medicinal fluid is discharged at a lower portion of the ringer solution input tube and a treatment solution, which is input into the treatment solution input tube, is prevented from flowing back toward the ringer solution input tube.

2. The medicinal fluid injector of claim 1, wherein the check value is formed at a center of a tubular body thereof with an expansion space, an edge protrusion protrudes downward from a top surface of the expansion space, a protrusion tube protrudes upward from a center of a bottom surface of the expansion space, a center of a packing installed in the expansion space is supported by the protrusion tube, and an edge of the packing fixedly adheres to the edge protrusion, so that an edge of the packing is bent downward to open a fluid path when pressure is applied downward from a top of the check valve, and the edge of the packing adheres to the edge protrusion to close the fluid path when pressure is applied upward from a bottom of the check valve.

3. The medicinal fluid injector of claim 2, wherein the protrusion tube is formed at an outer peripheral surface of an upper end thereof with an incised slot to form the fluid path of the check valve.

4. The medicinal fluid injector of claim 2, an opening hole is formed below blades, which support the protrusion tube, to form the fluid path of the check valve.
